(19) United States
(12) Patent Application Publication
(10) Pub. No.: US 2026/0301883 A1
(43) Pub. Date: Oct. 1, 2026

(54) PERSONALIZED DRUG COMPOUNDING BASED ON AI-DRIVEN DIAGNOSTICS AND REAL-TIME DATA INTEGRATION

(71) Applicant: Carol L Houston, Ostego, MO (US)

(72) Inventor: Carol L Houston, Ostego, MO (US)

(21) Appl. No.: 19/395,619

(22) Filed: Nov. 20, 2025

Related U.S. Application Data
(60) Provisional application No. 63/780,490, filed on Mar. 31, 2025.

(51) Int. Cl. G16C 20/50; G16C 20/70

(52) U.S. Cl. CPC G16C 20/50; G16C 20/70

(57) ABSTRACT

Methods, systems, and devices for personalized drug compounding based on artificial intelligence-driven diagnostics and real-time data integration are described. Some implementations may include a server configured to receive patient specimen and patient-specific clinical data, including biomarker expressions, pharmacogenomic profiles, and organ function metrics. The server may analyze the histopathology images to extract tissue subtypes and biomarker features, correlating these features with the clinical data to generate diagnostic outputs. Based on the diagnostic outputs and a clinical rule engine, the server may generate personalized drug compounding instructions specifying formulation types, dosages, and excipients tailored to the patient-specific clinical data. The server may update the compounding instructions in response to changes in the patient-specific clinical data, incorporating real-time adjustments to formulation types, dosages, or excipients to ensure precision and personalization in therapeutic interventions.

### CROSS REFERENCE TO RELATED APPLICATIONS

The present Application for Patent claims the benefit of U.S. Provisional application No. 63/780,490 entitled, Integrated Decision Support Platform for Personalized Drug Compounding Based on AI-Enhanced Histopathology and Biomarking Data, and which is hereby incorporated by reference in its entirety.

### FIELD OF TECHNOLOGY

The present disclosure relates generally to personalized medicine and automated pharmaceutical compounding. More particularly, it pertains to an AI-driven diagnostic-to-compounding platform (DSP) and, especially, to an AI engine subsystem within that platform for automatically analyzing patient diagnostic data and generating customized drug compounding instructions.

### BACKGROUND

The field of personalized medicine may involve the integration of advanced technologies to enhance diagnostic and therapeutic processes. Artificial intelligence may be utilized to analyze patient specimen, identifying disease subtypes and biomarkers that are relevant to disease characterization. Machine learning models may process large datasets to uncover patterns and correlations that inform clinical decision-making. Pharmacy compounding may involve the preparation of customized medications tailored to individual patient needs, often guided by established standards such as those outlined by the United States Pharmacopeia.

### SUMMARY

The described techniques relate to improved methods, systems, devices, and apparatuses for personalized drug compounding based on AI-driven diagnostics and real-time data integration. Some implementations may address these challenges by introducing an integrated decision support platform that combines artificial intelligence-enhanced specimen analysis with a dynamic formulation engine. Personalized drug therapy is an emerging approach in healthcare that aims to tailor medications to individual patient needs. Traditionally, after a diagnosis is made, medical professionals determine a treatment plan. This process can be time-consuming, prone to human error, and may not fully account for the breadth of available patient data (e.g., genetic information, real-time vital signs, complex drug interaction databases). Moreover, existing compounding practices often rely on standardized formulations that may not optimally suit every patient's unique physiology or condition.

This platform may leverage advanced machine learning models, multi-modal data fusion, and clinical rule engines to generate personalized drug compounding instructions in real time. By analyzing digitized histology slides and correlating the findings with patient-specific clinical data, the system may tailor medication formulations to align with individual therapeutic needs. The platform may dynamically adjust dosages, excipients, and delivery mechanisms based on evolving patient data, such as biomarker responses, pharmacogenomic profiles, and organ function metrics.

Some implementations may further ensure compliance with regulatory standards by incorporating automated checks against the workflow (for ex. United States Pharmacopeia Chapters 795, 797, and 800), as well as relevant Food and Drug Administration guidelines. This regulatory-aware functionality may enable seamless deployment in compounding pharmacy environments. Additionally, the platform may include mechanisms for explainable artificial intelligence, providing transparency and auditability for clinicians and regulatory bodies. By creating a closed-loop feedback system that may continuously refine therapeutic recommendations based on real-world patient outcomes, some implementations may represent a transformative advancement in precision medicine, bridging the gap between diagnostic pathology and personalized pharmaceutical compounding.

Therefore, an improved solution is needed to harness artificial intelligence for end-to-end personalization of drug therapy. Such a solution would analyze individual patient data (e.g., diagnosis outcomes, health records, biomarkers), determine an optimal medication formulation (including drug selection, dosage, and composition), and instruct compounding hardware to produce the personalized medicine, all while integrating with existing clinical workflows. It would reduce the burden on healthcare professionals, minimize errors, and potentially lead to better patient outcomes through more precise therapy.

### BRIEF DESCRIPTION OF THE DRAWINGS

A full and enabling description of the present disclosure, including the best mode thereof, directed to one of ordinary skill in the art, is set forth in the specification, which refers to the appended figures, in which:

FIG. 1 illustrates an example of a system for information processing configured for personalized drug compounding based on AI-driven diagnostics and real-time data integration in accordance with aspects of the present disclosure.

FIG. 2 shows an integrated decision platform for which supports techniques for personalized drug compounding based on AI-driven diagnostics and real-time data integration in accordance with various aspects of the present disclosure.

FIG. 3 illustrates an example of a process flow for personalized drug compounding based on tissues samples and bodily fluids AI-driven diagnostics and real-time data integration in accordance with various aspects of the present disclosure.

FIG. 4 shows a diagram of a system including a device for personalized drug compounding based on AI-driven diagnostics and real-time data integration in accordance with various aspects of the present disclosure.

FIG. 5 shows a flowchart illustrating a method for personalized drug compounding based on AI-driven diagnostics and real-time data integration in accordance with various aspects of the present disclosure.

FIG. 6 shows a flowchart illustrating a method for personalized drug compounding based on AI-driven diagnostics and real-time data integration in accordance with various aspects of the present disclosure.

### DETAILED DESCRIPTION

Reference will now be made in detail to present embodiments of the disclosure, one or more examples of which are illustrated in the accompanying drawings. The detailed description uses numerical and letter designations to refer to features in the drawings. Like or similar designations in the drawings and description have been used to refer to like or similar parts of the disclosure.

The word “exemplary” is used herein to mean “serving as an example, instance, or illustration.” Any implementation described herein as “exemplary” is not necessarily to be construed as preferred or advantageous over other implementations. Additionally, unless specifically identified otherwise, all embodiments described herein should be considered exemplary. As used herein, the terms “first”, “second”, and “third” may be used interchangeably to distinguish one component from another and are not intended to signify location or importance of the individual components. The terms “coupled,” “fixed,” “attached to,” and the like refer to both direct coupling, fixing, or attaching, as well as indirect coupling, fixing, or attaching through one or more intermediate components or features, unless otherwise specified herein. The singular forms “a”, “an”, and “the” include plural references unless the context clearly dictates otherwise. The term “at least one of” in the context of, e.g., “at least one of A, B, and C” refers to only A, only B, only C, or any combination of A, B, and C.

Approximating language, as used herein throughout the specification and claims, is applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 1, 2, 4, 10, 15, or 20 percent margin. These approximating margins may apply to a single value, either or both endpoints defining numerical ranges, and/or the margin for ranges between endpoints.

Methods, systems, devices, and apparatuses for personalized drug compounding based on AI-driven diagnostics and real-time data integration are disclosed. In some examples, current pharmacy compounding systems and diagnostic tools may operate in silos, creating inefficiencies and limiting the potential for fully personalized therapeutic interventions. Existing artificial intelligence-based pathology platforms may focus primarily on diagnostic interpretation, without directly linking their outputs to actionable pharmaceutical compounding instructions. Compounding systems may rely on static formulations that fail to account for evolving patient-specific factors such as genetic markers, biomarker responses, and organ function. This disconnect between diagnostic insights and therapeutic formulation may hinder the ability to deliver precision medicine. Additionally, the lack of real-time adaptability in compounding workflows and the absence of automated compliance checks with regulatory standards, such as those outlined in relevant pharmacopeia chapters, may further exacerbate these limitations. These challenges may underscore the need for an integrated solution that bridges the gap between pathology diagnostics

The present disclosure relates to a decision support platform (DSP) platform for patient-specific drug compounding and clinical decision support. The system integrates multimodal biomedical inputs, artificial intelligence (AI) analysis, regulatory logic, and patient-facing engagement into a unified architecture that supports automated, adaptive, and compliant pharmaceutical workflows.

Aspects of the disclosure are initially described in the context of networked computing systems and other aspects of the disclosure are further illustrated by and described with reference to apparatus diagrams, system diagrams, and flowcharts that relate to personalized drug compounding based on AI-driven diagnostics and real-time data integration.

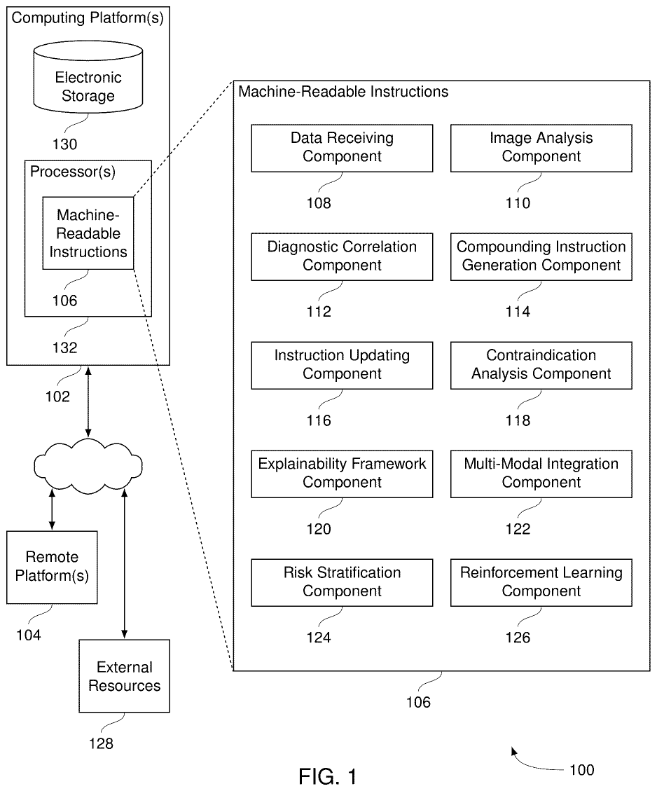

FIG. 1 illustrates an example of a system 100 for information processing configured for personalized drug compounding based on AI-driven diagnostics and real-time data integration in accordance with aspects of the present disclosure. For example, the system 100 may be configured for dynamically displaying information based on a context associated with a smart card, in accordance with one or more implementations. System 100 may include one or more computing platforms 102. Computing platform(s) 102 may be configured to communicate with one or more remote platforms 104 according to a client/server architecture, a peer-to-peer architecture, and/or other architectures. Remote platform(s) 104 may be configured to communicate with other remote platforms via computing platform(s) 102 and/or according to a client/server architecture, a peer-to-peer architecture, and/or other architectures. Users may access system 100 via remote platform(s) 104.

Computing platform(s) 102 may be configured by machine-readable instructions 106. Machine-readable instructions 106 may include one or more instruction components. The instruction components may include computer program components. The instruction components may include one or more of a data receiving component 108, an image analysis component 110, a diagnostic correlation component 112, a compounding instruction generation component 114, an instruction updating component 116, a contraindication analysis component 118, an explainability framework component 120, a multi-modal integration component 122, a risk stratification component 124, a reinforcement learning component 126 and/or other instruction components.

The data receiving component 108 may be configured as or otherwise support a means for receiving patient specimen and patient-specific clinical data including biomarker expressions, pharmacogenomic profiles, and organ function metrics. Patient Specimen can be any one of whole blood, tissue samples, urine, etc. In some implementations, the data receiving component 108 may accept histopathology images in various formats, such as DICOM or TIFF, to accommodate diverse imaging systems. The data receiving component 108 may include functionality to preprocess incoming images by adjusting resolution or color balance to align with analytical requirements.

The data receiving component 108 may be configured to handle patient-specific clinical data through secure channels, such as encrypted APIs, to maintain data integrity and confidentiality. In some implementations, the data receiving component 108 may integrate with electronic health record (EHR) systems to retrieve pharmacogenomic profiles and organ function metrics directly from patient records. The data receiving component 108 may support batch processing capabilities to manage large datasets efficiently, such as those generated during high-throughput diagnostic workflows.

The image analysis component 110 may be configured as or otherwise support a means for analyzing the patient specimen to extract tissue subtypes and biomarker features. In some implementations, the image analysis component 110 may determine tissue subtypes by segmenting regions of interest within the images based on color gradients and texture patterns. The image analysis component 110 may employ convolutional neural networks to identify biomarker features, such as HER2 or PD-L1 expression, by detecting specific staining intensities and distributions. In some implementations, the image analysis component 110 may analyze the spatial arrangement of cells to determine structural abnormalities indicative of disease progression. The image analysis component 110 may include preprocessing algorithms that adjust image contrast or normalize staining variations to align with analytical requirements. In some implementations, the image analysis component 110 may support multi-scale analysis to evaluate both cellular-level details and broader tissue architecture within the same image.

The diagnostic correlation component 112 may be configured as or otherwise support a means for correlating the extracted features with the patient-specific clinical data to generate diagnostic outputs. In some implementations, the diagnostic correlation component 112 may determine relationships between biomarker expression levels and pharmacogenomic profiles to identify potential therapeutic targets. In some implementations, the diagnostic correlation component 112 may cross-reference tissue subtype data with organ function metrics to assess compatibility with specific drug formulations. In some implementations, the diagnostic correlation component 112 may evaluate historical therapeutic response data alongside extracted histopathologic features to predict patient-specific treatment efficacy.

The compounding instruction generation component 114 may be configured as or otherwise support a means for generating personalized drug compounding instructions based on the diagnostic outputs and a clinical rule engine, the instructions may specify formulation types, dosages, and excipients tailored to the patient-specific clinical data. In some implementations, the compounding instruction generation component 114 may determine the appropriate formulation type, such as oral suspension, topical cream, or injectable solution, based on patient-specific factors like age, organ function, or therapeutic response history. In some implementations, the compounding instruction generation component 114 may specify dosages that align with pharmacogenomic profiles, adjusting for genetic markers that influence drug metabolism or efficacy. In some implementations, the compounding instruction generation component 114 may identify excipients that are compatible with patient allergy profiles, such as selecting lactose-free or preservative-free alternatives for individuals with sensitivities.

The instruction updating component 116 may be configured as or otherwise support a means for updating the personalized drug compounding instructions in response to changes in the patient-specific clinical data, the updates may incorporate real-time adjustments to the formulation types, dosages, or excipients. In some implementations, the instruction updating component 116 may determine adjustments to formulation types based on newly identified patient tolerances, such as switching from an injectable solution to a transdermal patch for patients with needle aversion. In some implementations, the instruction updating component 116 may incorporate changes to formulation types by considering updated organ function metrics, such as transitioning to a topical cream for patients with impaired renal function.

In some implementations, the instruction updating component 116 may determine dosage modifications in response to updated pharmacogenomic profiles, such as increasing the dosage for patients with genetic markers indicating rapid drug metabolism. In some implementations, the instruction updating component 116 may adjust dosages based on recent lab results, such as reducing the dosage for patients showing signs of drug toxicity. In some implementations, the instruction updating component 116 may determine excipient substitutions based on newly reported allergy profiles, such as replacing a preservative with a hypoallergenic alternative. In some implementations, the instruction updating component 116 may incorporate excipient changes by considering patient-specific dietary restrictions, such as selecting gluten-free excipients for individuals with celiac disease.

In some examples, the contraindication analysis component 118 may be configured as or otherwise support a means for analyzing the patient-specific clinical data to identify contraindications or adverse interactions among potential excipients, active pharmaceutical ingredients, or delivery mechanisms, and incorporating the identified contraindications or interactions into the generation of the personalized drug compounding instructions. In some implementations, the contraindication analysis component 118 may determine potential interactions between excipients and patient-specific allergy profiles, such as identifying lactose-based excipients that may trigger adverse reactions in lactose-intolerant individuals. In some implementations, the contraindication analysis component 118 may evaluate the compatibility of active pharmaceutical ingredients with existing medications in the patient's treatment regimen, such as identifying potential interactions between anticoagulants and nonsteroidal anti-inflammatory drugs. In some implementations, the contraindication analysis component 118 may assess the suitability of delivery mechanisms based on patient-specific conditions, such as determining whether an injectable formulation may pose risks for patients with bleeding disorders.

In some implementations, the contraindication analysis component 118 may analyze pharmacogenomic data to identify genetic markers that may influence adverse reactions to specific active pharmaceutical ingredients, such as identifying variations in CYP450 enzymes that may affect drug metabolism. In some implementations, the contraindication analysis component 118 may determine the potential for excipient interactions with dietary restrictions, such as identifying gluten-containing excipients for patients with celiac disease. In some implementations, the contraindication analysis component 118 may evaluate the risk of adverse interactions between delivery mechanisms and patient-specific organ function metrics, such as determining whether transdermal patches may be unsuitable for patients with compromised skin integrity.

In some examples, the explainability framework component 120 may be configured as or otherwise support a means for applying an explainable AI framework to the diagnostic outputs to generate interpretability data that may identify key features influencing the personalized drug compounding instructions, the interpretability data may be configured for review by clinicians or regulatory bodies. In some implementations, the explainability framework component 120 may determine the relative importance of specific biomarkers, such as HER2 or PD-L1, in influencing dosage recommendations. In some implementations, the explainability framework component 120 may generate visual representations, such as heatmaps, to highlight regions of histopathology images that contributed to the diagnostic outputs. In some implementations, the explainability framework component 120 may include textual summaries that describe how patient-specific clinical data, such as pharmacogenomic profiles, may have influenced the selection of excipients in the compounding instructions.

In some examples, the multi-modal integration component 122 may be configured as or otherwise support a means for integrating multi-modal data from histopathology images, structured clinical data, and unstructured electronic health record notes to enhance the diagnostic outputs and refine the personalized drug compounding instructions. In some implementations, the multi-modal integration component 122 may determine correlations between histopathology image features, such as tissue architecture, and structured clinical data, such as lab values or biomarker levels. In some implementations, the multi-modal integration component 122 may analyze unstructured electronic health record notes to extract relevant clinical observations, such as descriptions of patient symptoms or prior treatment responses, which may complement structured data inputs.

In some implementations, the multi-modal integration component 122 may incorporate natural language processing algorithms to interpret unstructured text data, such as physician notes, and convert it into structured formats for integration with other data types. In some implementations, the multi-modal integration component 122 may determine patterns across data modalities, such as linking genomic variations identified in structured clinical data with histopathologic features indicative of disease progression. In some implementations, the multi-modal integration component 122 may preprocess histopathology images to standardize resolution and color profiles, ensuring compatibility with structured and unstructured data inputs.

In some examples, the risk stratification component 124 may be configured as or otherwise support a means for conducting a risk stratification analysis on the patient-specific clinical data to classify the patient into a treatment response group, the classification may be used to adjust the formulation types, dosages, or excipients in the personalized drug compounding instructions. In some implementations, the risk stratification component 124 may determine treatment response groups based on biomarker expression levels, such as categorizing patients with high PD-L1 levels into groups that may benefit from specific immunotherapy formulations. In some implementations, the risk stratification component 124 may classify patients into treatment response groups by analyzing pharmacogenomic profiles, such as grouping individuals with CYP450 or other specific genetic variants that may influence drug metabolism into distinct dosage categories. In some implementations, the risk stratification component 124 may determine treatment response groups by evaluating organ function metrics, such as assigning patients with impaired renal function to groups that may require adjusted excipient compositions.

In some examples, the reinforcement learning component 126 may be configured as or otherwise support a means for incorporating reinforcement learning to evaluate longitudinal patient outcomes and dynamically update the clinical rule engine, the updates may be used to refine the personalized drug compounding instructions based on observed therapeutic performance. In some implementations, the reinforcement learning component 126 may determine reward functions based on specific clinical metrics, such as biomarker normalization or symptom reduction over time. In some implementations, the reinforcement learning component 126 may evaluate patient adherence data to adjust the weighting of certain clinical parameters in the rule engine. In some implementations, the reinforcement learning component 126 may incorporate feedback from clinicians regarding observed patient responses to further refine the decision-making process.

In some implementations, computing platform(s) 102, remote platform(s) 104, and/or external resources 128 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which computing platform(s) 102, remote platform(s) 104, and/or external resources 128 may be operatively linked via some other communication media.

A given remote platform may include one or more processors configured to execute computer program components. The computer program components may be configured to enable an expert or user associated with the given remote platform to interface with system 100 and/or external resources 128, and/or provide other functionality attributed herein to remote platform(s) 104. By way of non-limiting example, a given remote platform and/or a given computing platform may include one or more of a smart card, a server, a desktop computer, a laptop computer, a handheld computer, a tablet computing platform, a NetBook, a Smartphone, a gaming console, and/or other computing platforms. In some implementations, the computing platform(s) 102 may comprise server(s), and the remote platform(s) 104 may comprise remotely located client computing platform(s).

External resources 128 may include sources of information outside of system 100, external entities participating with system 100, and/or other resources. In some implementations, some or all the functionality attributed herein to external resources 128 may be provided by resources included in system 100.

Computing platform(s) 102 may include electronic storage 130, one or more processors 132, and/or other components. Computing platform(s) 102 may include communication lines, or ports to enable the exchange of information with a network and/or other computing platforms. Illustration of computing platform(s) 102 in FIG. 1 is not intended to be limiting. Computing platform(s) 102 may include a plurality of hardware, software, and/or firmware components operating together to provide the functionality attributed herein to computing platform(s) 102. For example, computing platform(s) 102 may be implemented by a cloud of computing platforms operating together as computing platform(s) 102.

Electronic storage 130 may comprise non-transitory storage media that electronically stores information. The electronic storage media of electronic storage 130 may include one or both of system storage that is provided integrally (i.e., substantially non-removable) with computing platform(s) 102 and/or removable storage that is removably connectable to computing platform(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from computing platform(s) 102, information received from remote platform(s) 104, and/or other information that enables computing platform(s) 102 to function as described herein.

Processor(s) 132 may be configured to provide information processing capabilities in computing platform(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute components 108, 110, 112, 114, 116, 118, 120, 122, 124, 126, and/or other components. Processor(s) 132 may be configured to execute components 108, 110, 112, 114, 116, 118, 120, 122, 124, 126, and/or other components by software; hardware; firmware; some combination of software, hardware, and/or firmware; and/or other mechanisms for configuring processing capabilities on processor(s) 132. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, 114, 116, 118, 120, 122, 124, and/or 126 are illustrated in FIG. 1 as being implemented within a single processing unit, in implementations in which processor(s) 132 includes multiple processing units, one or more of components 108, 110, 112, 114, 116, 118, 120, 122, 124, and/or 126 may be implemented remotely from the other components. The description of the functionality provided by the different components 108, 110, 112, 114, 116, 118, 120, 122, 124, and/or 126 described below is for illustrative purposes, and is not intended to be limiting, as any of components 108, 110, 112, 114, 116, 118, 120, 122, 124, and/or 126 may provide more or less functionality than is described. For example, one or more of components 108, 110, 112, 114, 116, 118, 120, 122, 124, and/or 126 may be eliminated, and some or all of its functionality may be provided by other ones of components 108, 110, 112, 114, 116, 118, 120, 122, 124, and/or 126. As another example, processor(s) 132 may be configured to execute one or more additional components that may perform some or all the functionality attributed below to one of components 108, 110, 112, 114, 116, 118, 120, 122, 124, and/or 126.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

The next figure is shown to have specific input into each block, however one should appreciate that while a specific arrow is shown in the figure, it would not depart from the scope of the invention to have another input not shown.

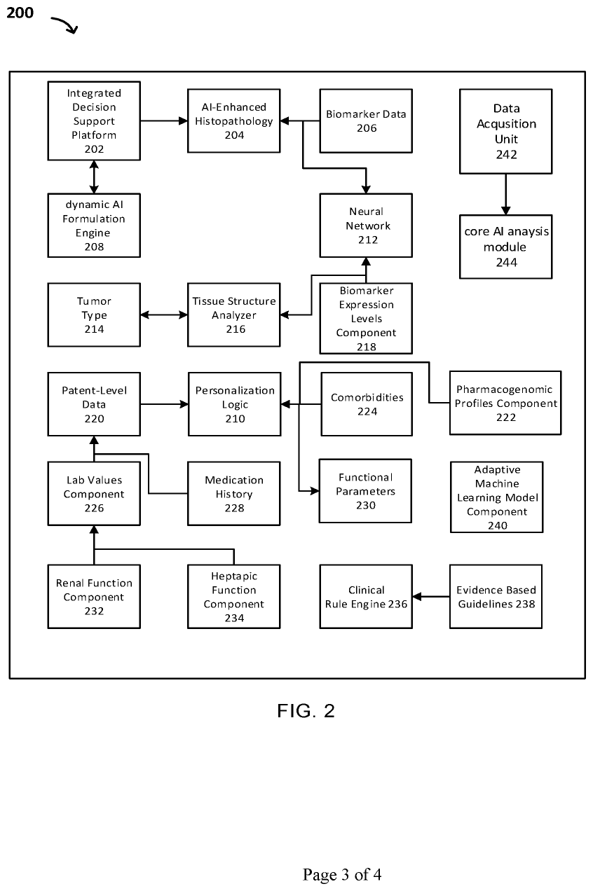

As depicted in FIG. 2, integrated decision platform 200 may include one or more of an integrated decision support platform 202, AI-enhanced histopathology 204, biomarker data 206, a dynamic AI formulation engine 208, personalization logic 210, a neural network 212, a tumor type 214, tissue structure analyzer 216, a biomarker expression levels component 218, patient-level data 220, pharmacogenomic profiles component 222, a comorbidities 224, a lab values component 226, medication history 228, functional parameters 230, a renal function component 232, a hepatic function component 234, a clinical rule engine 236, evidence-based guidelines 238, an adaptive machine learning models component 240, and/or other components.

The integrated decision support platform 202 may include mechanisms for personalized drug compounding based on AI-driven diagnostics and real-time data integration. The integrated decision support platform 202 may be a cloud-based system designed to process and analyze diverse clinical and diagnostic data. The platform may incorporate secure infrastructure to allow remote access by clinicians while maintaining audit trail integrity. The integrated decision support platform 202 may interact with other components, such as the AI-enhanced histopathology 204 and the dynamic AI formulation engine 208, to generate personalized therapeutic recommendations. In some implementations, the integrated decision support platform 202 may include modules for compliance checks with applicable Federal and state requirements.

The AI-enhanced histopathology 204 may provide analysis of digitized histology slides to identify tissue subtypes and detect biomarker expressions. The AI-enhanced histopathology 204 may utilize a trained neural network to extract clinically significant features from pathological images. The system may analyze morphological patterns, such as tissue structure and tumor type, to support diagnostic accuracy. The AI-enhanced histopathology 204 may work in conjunction with the biomarker data 206 to identify relevant expressions that inform therapeutic decisions. In some implementations, the AI-enhanced histopathology 204 may include explainability tools, such as SHAP or LIME, to support regulatory compliance and clinician trust.

The biomarker data 206 may represent clinically significant features relevant to disease characterization and therapeutic decision-making. The biomarker data 206 may include quantitative or qualitative information about expressions such as HER2, PD-L1, and EGFR. The data may be derived from histopathologic analysis and integrated with patient-level data 220 to inform personalized drug compounding. The biomarker data 206 may be used by the dynamic AI formulation engine 208 to adjust dosing and ingredient ratios in compounded medications. In some implementations, the biomarker data 206 may be stored in a curated clinical database for cross-referencing with evidence-based guidelines 238.

The dynamic AI formulation engine 208 may determine personalized medication compounding instructions based on patient-specific clinical factors. The dynamic AI formulation engine 208 may use adaptive machine learning models 240 to refine formulation outputs based on real-world performance data. The engine may generate instructions for adjusting formulation types, dosages, and excipients to align with individual patient needs. The dynamic AI formulation engine 208 may interact with the clinical rule engine 236 to apply evidence-based guidelines 238 during the compounding process. In some implementations, the dynamic AI formulation engine 208 may include a feedback loop to update recommendations as patient data evolves.

The personalization logic 210 may adjust formulation types, dosages, and excipients to align with individual patient data. The personalization logic 210 may integrate inputs from pharmacogenomic profiles 222, comorbidities 224, and functional parameters 230 to tailor therapeutic recommendations. The logic may dynamically update compounding protocols based on changes in patient-level data 220, such as lab values 226 or medication history 228. The personalization logic 210 may work in conjunction with the dynamic AI formulation engine 208 to ensure that formulations are patient-specific. In some implementations, the personalization logic 210 may include algorithms for dose titration and excipient modification.

The neural network 212 may analyze pathological images to extract features such as tumor type, tissue structure, and biomarker expression levels. The neural network 212 may be trained on a dataset of digitized histology slides to identify clinically relevant patterns. The extracted features may be used to inform the AI-enhanced histopathology 204 and biomarker data 206 components. The neural network 212 may interact with the multi-modal data fusion engine to combine image-based features with structured clinical data. In some implementations, the neural network 212 may include mechanisms for continuous learning to improve diagnostic precision.

The tumor type 214 may include classifications derived from histopathologic analysis to inform therapeutic recommendations. The tumor type 214 may be identified based on morphological patterns and biomarker expressions detected in histology slides. The classifications may be used by the dynamic AI formulation engine 208 to determine appropriate compounding protocols. The tumor type 214 may be cross-referenced with evidence-based guidelines 238 to ensure alignment with validated treatment protocols. In some implementations, the tumor type 214 may include subtypes relevant to oncology and rare diseases.

The tissue structure 216 may represent morphological patterns identified in histology slides for diagnostic and compounding purposes. The tissue structure 216 may include features such as cellular arrangement, density, and architectural organization. The identified patterns may be analyzed by the AI-enhanced histopathology 204 to support disease characterization. The tissue structure 216 may be integrated with biomarker data 206 to provide a comprehensive diagnostic profile. In some implementations, the tissue structure 216 may be used to stratify patients into risk or treatment response clusters.

The biomarker expression levels 218 may include quantitative or qualitative data relevant to disease progression and treatment response. The biomarker expression levels 218 may be determined through AI-driven analysis of histopathologic images. The data may inform the dynamic AI formulation engine 208 in generating personalized compounding instructions. The biomarker expression levels 218 may be cross-referenced with pharmacogenomic profiles 222 to identify potential drug interactions or contraindications. In some implementations, the biomarker expression levels 218 may include metrics for monitoring therapeutic efficacy over time.

The patient-level data 220 may include clinical information such as genetic markers, organ function, allergy profiles, and therapeutic response history. The patient-level data 220 may be collected from electronic health records, lab results, and patient-reported outcomes. The data may be used by the personalization logic 210 to tailor drug formulations to individual needs. The patient-level data 220 may interact with the clinical rule engine 236 to ensure compliance with evidence-based guidelines 238. In some implementations, the patient-level data 220 may include longitudinal records to track changes in clinical status.

The pharmacogenomic profiles 222 may provide insights into genetic factors influencing drug metabolism and efficacy. The pharmacogenomic profiles 222 may include information about genetic variants that affect enzyme activity, drug transport, and receptor binding. The profiles may be used by the dynamic AI formulation engine 208 to adjust dosing and ingredient selection. The pharmacogenomic profiles 222 may be integrated with biomarker data 206 to create a comprehensive therapeutic profile. In some implementations, the pharmacogenomic profiles 222 may include data from next-generation sequencing or genotyping assays.

The comorbidities 224 may include concurrent medical conditions that impact therapeutic decisions and compounding protocols. The comorbidities 224 may be identified through patient-level data 220 and clinical assessments. The information may be used by the personalization logic 210 to adjust formulation types and dosages. The comorbidities 224 may interact with lab values 226 to determine potential contraindications or drug interactions. In some implementations, the comorbidities 224 may include chronic conditions such as diabetes, hypertension, or autoimmune disorders.

The lab values 226 may represent diagnostic metrics that inform dosing and ingredient ratios in compounded medications. The lab values 226 may include parameters such as blood counts, electrolyte levels, and organ function tests. The data may be used by the dynamic AI formulation engine 208 to generate personalized compounding instructions. The lab values 226 may interact with pharmacogenomic profiles 222 to identify potential risks or adjustments in therapy. In some implementations, the lab values 226 may include real-time updates to reflect changes in patient status.

The medication history 228 may include records of prior treatments to guide personalized drug formulation. The medication history 228 may provide information about previously prescribed drugs, dosages, and treatment durations. The data may be used by the personalization logic 210 to avoid potential drug interactions or redundant therapies. The medication history 228 may interact with patient-level data 220 to create a comprehensive therapeutic profile. In some implementations, the medication history 228 may include data from pharmacy records or patient-reported outcomes.

The functional parameters 230 may represent clinical metrics such as organ function relevant to therapeutic compounding. The functional parameters 230 may include data on renal function 232 and hepatic function 234. The metrics may be used by the dynamic AI formulation engine 208 to adjust dosing and ingredient selection. The functional parameters 230 may interact with lab values 226 to provide a detailed assessment of patient health. In some implementations, the functional parameters 230 may include additional metrics such as cardiac function or respiratory capacity.

The renal function 232 may include data on kidney performance to inform dosing adjustments and ingredient selection. The renal function 232 may be assessed through lab values 226 such as creatinine clearance or glomerular filtration rate. The data may be used by the personalization logic 210 to tailor drug formulations to individual needs. The renal function 232 may interact with pharmacogenomic profiles 222 to identify potential risks related to drug metabolism. In some implementations, the renal function 232 may include longitudinal data to monitor changes over time.

The hepatic function 234 may represent liver performance metrics relevant to drug metabolism and formulation. The hepatic function 234 may be assessed through lab values 226 such as liver enzyme levels or bilirubin concentrations. The data may be used by the dynamic AI formulation engine 208 to adjust dosing and ingredient ratios. The hepatic function 234 may interact with patient-level data 220 to provide a comprehensive assessment of metabolic capacity. In some implementations, the hepatic function 234 may include additional metrics such as albumin levels or coagulation factors.

The clinical rule engine 236 may apply evidence-based guidelines to generate therapeutic recommendations. The clinical rule engine 236 may include a database of validated protocols for drug compounding and therapeutic decision-making. The engine may interact with the dynamic AI formulation engine 208 to ensure compliance with regulatory standards. The clinical rule engine 236 may use adaptive machine learning models 240 to refine recommendations based on real-world data. In some implementations, the clinical rule engine 236 may include modules for automated compliance checks with applicable Federal and state requirements.

The evidence-based guidelines 238 may include validated protocols for drug compounding and therapeutic decision-making. The evidence-based guidelines 238 may be derived from clinical studies, regulatory standards, and expert consensus. The guidelines may be used by the clinical rule engine 236 to generate personalized therapeutic recommendations. The evidence-based guidelines 238 may interact with patient-level data 220 to ensure alignment with individual clinical needs. In some implementations, the evidence-based guidelines 238 may include updates to reflect new research or regulatory changes.

The adaptive machine learning models 240 may refine formulation outputs based on accumulated real-world performance data. The adaptive machine learning models 240 may use algorithms to analyze longitudinal patient outcomes and adjust therapeutic recommendations. The models may interact with the dynamic AI formulation engine 208 to update compounding protocols in real time. The adaptive machine learning models 240 may include mechanisms for reinforcement learning to improve dosing strategies. In some implementations, the adaptive machine learning models 240 may incorporate unsupervised clustering to identify treatment response patterns.

In some implementations, the integrated decision support platform 202 may connect AI-enhanced histopathology 204 and biomarker data 206 to the dynamic AI formulation engine 208, which may interact with personalization logic 210 to process patient-specific inputs. The neural network 212 may analyze tumor type 214, tissue structure 216, and biomarker expression levels 218 to extract clinically significant features, which may then be combined with patient-level data 220, including pharmacogenomic profiles 222, comorbidities 224, lab values 226, medication history 228, and functional parameters 230 such as renal function 232 and hepatic function 234.

In some implementations, the clinical rule engine 236 may apply evidence-based guidelines 238 and adaptive machine learning models 240 to dynamically adjust therapeutic recommendations. The dynamic AI formulation engine 208 may integrate outputs from the neural network 212 and patient-level data 220 to determine precise dosing, ingredient ratios, and delivery methods. The personalization logic 210 may continuously refine these outputs based on updated patient data, such as lab values 226 or biomarker expression levels 218, ensuring alignment with evolving clinical factors. I

It should be understood that whole blood or other bodily fluids can be analyzed using the same techniques presented herein without departing from the scope of the invention. The invention's technical architecture comprises several components working in concert.

As also shown in FIG. 2, first a data acquisition unit 242 obtains raw data from patient samples. This data can include numerical laboratory results (e.g., complete blood count values, chemistry panels, genetic marker readings) and/or digital images of blood smears or cells. For example, a microscopic image of a stained blood smear can be captured and fed into the system. The core AI analysis module 242 then processes this data. This module may employ machine learning models, such as neural network 212 from FIG. 2 trained on hematological data, to identify patterns and abnormalities in the blood sample. Key indicators of blood disorders are detected by the AI with high accuracy, such as abnormal cell morphology, counts, or marker levels. Indeed, AI algorithms have demonstrated the ability to recognize blood cell anomalies and classify diseases like anemia, leukemia, sickle cell disease, and thrombocytopenia from blood samples. The difference between the AI enhanced histopathology 204 and the core AI analysis module 244 is that the one analyzes tissue data analyzes bodily fluids.

In the context of this invention, the AI analysis module 244 leverages such capabilities to provide an automated, detailed analysis of the sample. It can, for instance, identify the presence of leukemia blast cells or irregularly shaped sickle erythrocytes, flag abnormal platelet clumping, or interpret atypical patterns in a patient's routine blood test results. The module may combine multiple data sources, laboratory parameters from the lab values component 226, imaging, even genomic data, which is crucial since effective hematologic diagnosis often depends on integrating diverse data sources (imaging, pathology, omics, lab tests) in a unified analysis. By handling this integration via machine intelligence, the system ensures no relevant detail is overlooked, thus improving diagnostic accuracy and consistency. Notably, the AI analysis can also incorporate patient-specific historical data 226 (previous test results, medical history) to enhance context. This allows it to perform risk stratification and predictions, such as forecasting disease progression or complication risks, which further inform the treatment strategy.

Once the blood sample analysis is complete, the treatment recommendation engine automatically formulates a personalized drug compounding plan. This component is effectively an AI-driven clinical decision support system that translates the diagnostic findings into a therapeutic prescription tailored for the individual. The engine contains or accesses a knowledge base of blood disorder treatments, including standard medications, dosages, combination therapy protocols, and compounding formulas known in hematology. It also utilizes patient-specific factors (for example, the patient's age, weight, genetic polymorphisms affecting drug metabolism, allergies, and comorbid conditions) when devising a treatment. Using either rule-based algorithms or predictive models (or a hybrid of both), the system matches the identified hematologic condition(s) with optimal treatment ingredients and strengths.

Importantly, the output is not limited to selecting a single existing drug. Instead, the system can propose a compounded medication regimen composed of multiple agents if warranted. For instance, if the AI analysis determines that a patient has iron-deficiency anemia with an underlying vitamin B_12 deficiency, the engine might recommend a combined treatment course. For example, a compounded supplement containing appropriate dosages of iron, vitamin B_12, and folic acid-rather than three separate prescriptions. For a more complex example, consider a patient with a certain subtype of leukemia. The AI may identify specific genetic markers or cell characteristics from the blood that suggest the patient will respond best to a particular chemotherapy combination. The system can then present a personalized chemotherapy cocktail, specifying two or three drugs to be mixed in precise proportions, aligned with that subtype and even adjusted to the patient's metabolism. This level of personalization is facilitated by AI's ability to consider how individual patients respond to different drugs. In fact, recent advances in AI for hematology have shown that algorithms can predict patient-specific drug responses and optimal regimens (for example, selecting the most effective anticoagulant and dose by analyzing a patient's genomic data and clotting metrics). Harnessing these capabilities, the invention's treatment engine ensures that the recommended compounded therapy is evidence-based and tailored, it might suggest dosing adjustments that maximize efficacy while minimizing side effects, guided by patterns learned from clinical data.

After determining the optimal drug combination, the system then presents the personalized compounding treatment plan to healthcare providers (and optionally to patients, in a user-friendly summary). The plan can be displayed via a graphical user interface or generated as a report on an application or on a user device. It typically includes, key findings from the sample analysis that led to that conclusion and recommended treatment formulation. For example, the “presence of sickle-shaped cells, hemoglobin S detected, low hemoglobin level” or “abnormal blast cell count indicating acute myelogenous leukemia”.

Additionally, the system might output a directive such as “Compounded Therapeutic Formula A: contains Drug X (Y mg/mL)+Drug Z (W mg/mL) in 100 mL saline; administer IV over N hours, daily for 5 days”. It would also list any supportive treatments if applicable (maybe an adjuvant like a drug to mitigate side effects). For disorders requiring long-term management, the recommendation might include a personalized dosing schedule or titration plan. A titration plan is a structured approach used in quantitative chemical analysis to determine the concentration of a specific substance (analyte) in a solution by slowly adding a reagent of known concentration (titrant) until a reaction endpoint is reached.

If the system is linked to a compounding pharmacy apparatus or software, it could even transmit the formula instructions directly for preparation. This invention thus not only advises which drugs to use but also in what combination and dosage, effectively generating a tailor-made prescription for the pharmacist to fill. The compounding aspect is critical for conditions where no off-the-shelf medication fits all needs. For example, pediatric blood disorder patients often require custom-dosed formulations, and this system can automatically produce those specifications. Furthermore, the system's knowledge base and algorithms ensure that any proposed combination adheres to safety and pharmaceutical compounding guidelines (e.g., checking that the active ingredients are compatible and stable together, and that the dosage is within accepted therapeutic limits).

The invention is applicable to a broad range of physiological and pathological conditions and can be used with multiple bodily fluid sample types. It is particularly useful with blood-related disorders, encompassing both non-malignant and malignant hematologic conditions. Non-malignant examples include various forms of anemia (iron-deficiency anemia, pernicious anemia due to B_12 deficiency, hemolytic anemia), clotting and bleeding disorders (such as hemophilia, von Willebrand disease, or hypercoagulable states like Factor V Leiden thrombophilia), and disorders of blood cell counts like thrombocytopenia or polycythemia. Malignant or proliferative conditions include leukemias, lymphomas (as far as they have blood biomarkers), multiple myeloma, and myelodysplastic syndromes. For each of these, the system's AI analysis module is configured to detect the hallmark indicators. For instance, in suspected leukemia cases, it will analyze white blood cell morphology and counts, possibly flagging the presence of blasts or abnormal lymphocytes, it might leverage AI-driven flow cytometry interpretations to classify leukemia subtypes. In clotting disorders, as another example, the system could analyze coagulation test results and genetic factors to assess thrombosis risk and then recommend a personalized anticoagulation plan (e.g., tailoring warfarin dosage or suggesting a specific DOAC (Direct Oral Anti-Coagulant) along with a compounded titration schedule).

In the case of anemias, the AI might determine whether the cause is nutritional deficiency, chronic disease, or a genetic condition like thalassemia by looking at patterns in the complete blood count and iron studies, and then suggest the correct therapeutic agents (iron supplements, vitamins, or in the case of thalassemia, perhaps chelation therapy for iron overload or even gene therapy candidates, depending on the scope of the system's data). Sickle cell disease provides an illustrative use case. The AI can detect the abnormal hemoglobin forms from lab results or even identify sickle-shaped cells on a smear, confirm the diagnosis, and then propose a treatment regimen. This might include a compounded prescription for hydroxyurea (a drug commonly used to reduce sickling crises) in a patient-specific dose combined with folic acid supplementation and an appropriate pain management protocol, thereby giving a comprehensive plan.

Similarly, for hematological cancers, once the AI classifies the cancer subtype, the treatment engine may suggest a particular chemotherapy or immunotherapy cocktail (for example, combining two chemotherapeutic drugs and a monoclonal antibody, at doses calculated per the patient's body surface area and genetic drug sensitivity). The ability of AI to help guide therapeutic decisions in diseases like leukemia, lymphoma, sickle cell disease, and thrombosis has been noted in medical literature, and this invention operationalizes that guidance by directly outputting a proposed therapy regimen. The invention is applicable to a wide spectrum of clinical use cases and is compatible with multiple biological fluid matrices, including blood-based and non-blood-based samples (e.g., urine, saliva, cerebrospinal fluid, synovial fluid, and similar specimens).

A notable aspect of this invention is the emphasis on personalized drug compounding. Unlike conventional decision-support systems which might simply select a drug from a list, this system can design a combination therapy that is custom-tailored. It effectively acts as an AI-assisted pharmacist in addition to being a diagnostician. The term “compounding” here implies that the recommended treatment could involve mixing specific ingredients in customized ratios or forms, something commonly done in compounding pharmacies for individualized patient needs. For example, if the optimal treatment for a certain blood disorder requires properties from multiple existing drugs (say Drug A and Drug B), the system can suggest using both in a combined formulation. This may be especially useful for rare blood disorders or unique patient cases where standard single-drug therapy is suboptimal. The system ensures evidence-based compounding decisions by drawing on clinical data and guidelines: it knows, for instance, what drug combinations have been effective for similar patients in the past (through its machine learning training or knowledge database) and what combinations are safe to prepare together.

This approach aligns with the trend of AI in pharmacy to yield “tailored medication solutions” via intelligent decision support. The output to the user (typically a clinician or pharmacist) is a clear set of instructions for creating and administering the therapy. In practice, the clinician could review this AI-generated plan and, if acceptable, authorize the compounding of the medication as recommended. The invention may also present alternative options in some cases, ranked by predicted efficacy, giving the medical professional flexibility to choose. The user interface might highlight the rationale for the AI's recommendation—for example, “Patient exhibits marker X, which research shows responds well to Drug A; however, due to patient's Y deficiency, adding Drug B is advised to prevent complications. Therefore, a combined formulation is recommended.” In doing so, the system provides transparency and confidence in its suggestions, functioning as a collaboration between AI and physician rather than a black-box directive.

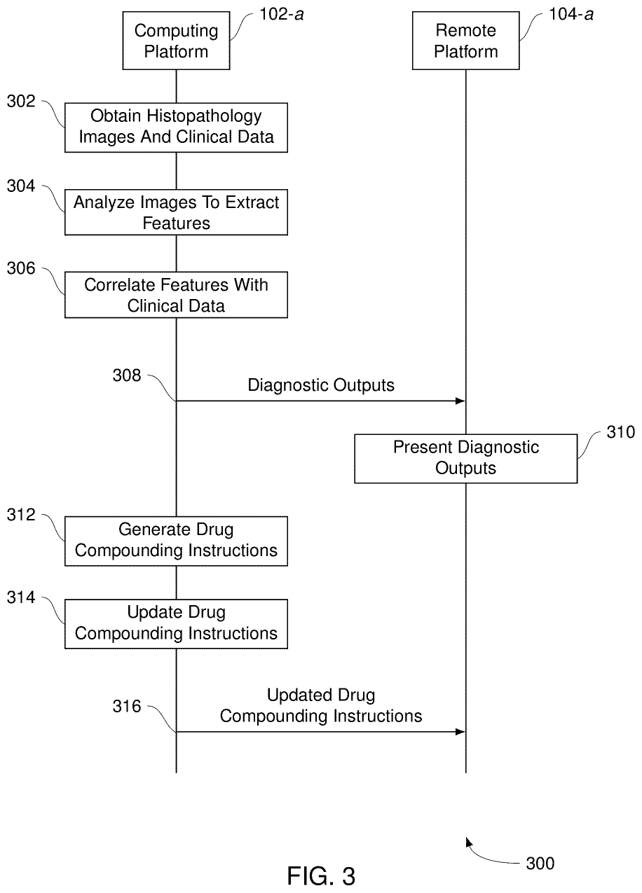

FIG. 3 illustrates an example of a process flow 300 for personalized drug compounding based on tissue samples and AI-driven diagnostics and real-time data integration in accordance with aspects of the present disclosure. In some examples, the process flow 300 may implement aspects of the system 100. For example, the process flow 300 may include a computing platform 102-a (e.g., a server) and a remote platform 104-a (e.g., a user device), which may be examples of corresponding devices described herein. In some implementations, a computing platform 102-a receives patient specimen and patient-specific clinical data, analyzes the images of the tissue samples and data to generate diagnostic outputs, formulates personalized drug compounding instructions based on the outputs, and updates these instructions in real-time, while a remote platform 104-a facilitates user interaction with the personalized instructions and diagnostic results.

At 302, the computing platform 102-a may obtain patient specimen and patient-specific clinical data including biomarker expressions, pharmacogenomic profiles, and organ function metrics. For example, the computing platform 102-a may access patient specimen that include annotations highlighting tissue subtypes and biomarker expression levels such as HER2 or PD-L1. In some implementations, the computing platform 102-a may retrieve patient-specific clinical data from a remote platform 104-a, which may include allergy profiles, therapeutic response history, or lab values related to renal and hepatic function.

At 304, the computing platform 102-a may analyze the patient specimen to extract tissue subtypes and biomarker features. For example, the computing platform 102-a may identify histological patterns such as glandular structures or stromal components that may correspond to specific tissue subtypes. In some implementations, the computing platform 102-a may detect biomarker features such as HER2 amplification or PD-L1 expression levels that may indicate therapeutic targets. In other implementations, the computing platform 102-a may apply machine learning models to determine additional features, such as tumor grade or necrotic regions, that may contribute to clinical decision-making.

At 306, the computing platform 102-a may correlate the extracted features with the patient-specific clinical data to generate diagnostic outputs. For example, the computing platform 102-a may match biomarker expression levels such as HER2 or PD-L1 with pharmacogenomic profiles to determine potential therapeutic targets. In some implementations, the computing platform 102-a may cross-reference tissue subtypes with curated clinical databases to identify evidence-based treatment guidelines relevant to the extracted features. In other implementations, the computing platform 102-a may integrate allergy profiles and organ function metrics with histopathologic findings to adjust diagnostic outputs for patient-specific tolerability considerations.

At 308, the computing platform 102-a may transmit the diagnostic outputs to the remote platform 104-a. For example, the computing platform 102-a may send annotated histopathology images that may include highlighted tissue subtypes and biomarker expression levels such as HER2 or PD-L1 to the remote platform 104-a for clinician review. In some implementations, the computing platform 102-a may transmit a summary report that may include correlations between extracted features and patient-specific clinical data, such as pharmacogenomic profiles or organ function metrics, to the remote platform 104-a. In other implementations, the computing platform 102-a may deliver real-time alerts to the remote platform 104-a, which may notify clinicians of potential therapeutic targets or contraindications based on the diagnostic outputs.

At 310, the remote platform 104-a may present the diagnostic outputs to facilitate user interaction. For example, the remote platform 104-a may display annotated histopathology images that may include highlighted tissue subtypes and biomarker expression levels such as HER2 or PD-L1. In some implementations, the remote platform 104-a may generate interactive dashboards that may allow clinicians to explore correlations between extracted features and patient-specific clinical data, such as pharmacogenomic profiles or organ function metrics. In other implementations, the remote platform 104-a may provide filtering options that may enable users to focus on specific diagnostic outputs, such as tissue subtypes or biomarker features relevant to therapeutic decision-making.

At 312, the computing platform 102-a may generate personalized drug compounding instructions based on the diagnostic outputs and a clinical rule engine, the instructions specifying formulation types, dosages, and excipients tailored to the patient-specific clinical data. For example, the computing platform 102-a may determine that a patient with HER2-positive biomarker expression may require a specific injectable formulation with adjusted excipient ratios to enhance tolerability. In some implementations, the computing platform 102-a may generate instructions for an oral suspension tailored to a pediatric patient's weight and allergy profile, incorporating alternative excipients to avoid known allergens. In other implementations, the computing platform 102-a may specify a transdermal delivery method for a patient with compromised renal function, adjusting the dosage to account for reduced clearance rates.

At 314, the computing platform 102-a may update the personalized drug compounding instructions in response to changes in the patient-specific clinical data, the updates incorporating real-time adjustments to the formulation types, dosages, or excipients. For example, the computing platform 102-a may adjust the dosage of a compounded medication for a patient whose updated lab results indicate reduced renal function, tailoring the formulation to mitigate potential toxicity. In some implementations, the computing platform 102-a may modify the excipient composition for a patient who develops a new allergy, substituting an alternative ingredient to maintain compatibility with the patient's updated allergy profile. In other implementations, the computing platform 102-a may transition the formulation type from an injectable to an oral suspension based on new clinical data indicating improved swallowing ability in the patient.

At 316, the computing platform 102-a may transmit the updated personalized drug compounding instructions to the remote platform 104-a. For example, the computing platform 102-a may send a detailed formulation report specifying adjusted dosages and excipient compositions tailored to the patient's updated clinical data. In some implementations, the computing platform 102-a may transmit a notification to the remote platform 104-a, which may include a summary of the changes made to the compounding instructions, such as a shift from an injectable formulation to an oral suspension. In other implementations, the computing platform 102-a may deliver a secure file containing the updated instructions, which may include annotations highlighting the rationale for specific adjustments based on recent biomarker or lab data.

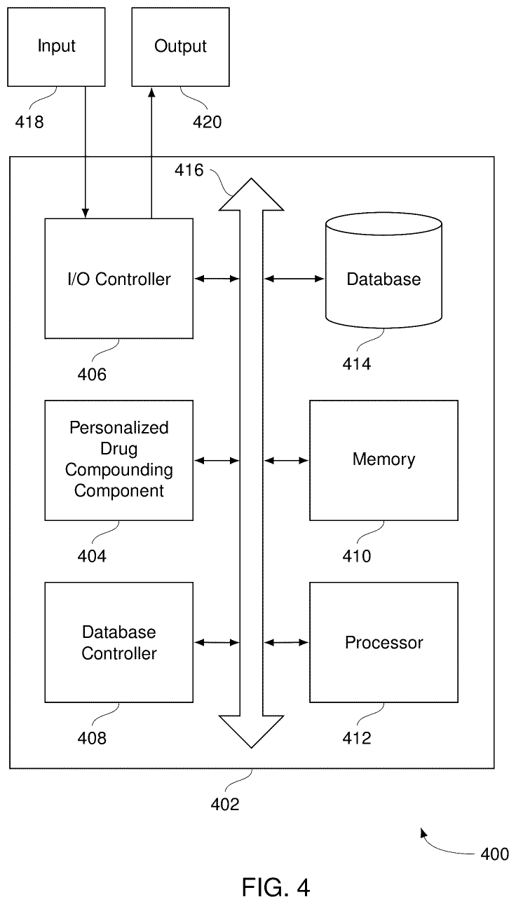

FIG. 4 shows a diagram of a system 400 including a device 402 configured for personalized drug compounding based on AI-driven diagnostics and real-time data integration in accordance with aspects of the present disclosure. The device 402 may be an example of or include the components of a computing platform 102 and a remote platform 104 as described herein. The device 402 may include components for bi-directional data communications including components for transmitting and receiving communications, including a personalized drug compounding component 404, an I/O controller 406, a database controller 408, memory 410, a processor 412, and a database 414. These components may be in electronic communication via one or more buses (e.g., bus 416).

The personalized drug compounding component 404 may be an example of one or more components of the system 100 as described herein. For example, the personalized drug compounding component 404 may perform any of the methods or processes described above with reference to machine-readable instructions 106 in connection with FIG. 1. In some cases, the personalized drug compounding component 404 may be implemented in hardware, software executed by a processor, firmware, or any combination thereof.

The I/O controller 406 may manage input signals 418 and output signals 4128 for the device 402. The I/O controller 406 may also manage peripherals not integrated into the device 402. In some cases, the I/O controller 406 may represent a physical connection or port to an external peripheral. In some cases, the I/O controller 406 may utilize an operating system such as iOS®, ANDROID®, MS-DOS®, MS-WINDOWS®, OS/2®, UNIX®, LINUX®, or another known operating system. In other cases, the I/O controller 406 may represent or interact with a modem, a keyboard, a mouse, a touchscreen, or a similar device. In some cases, the I/O controller 406 may be implemented as part of a processor. In some cases, a user may interact with the device 402 via the I/O controller 406 or via hardware components controlled by the I/O controller 406.

The database controller 408 may manage data storage and processing in a database 414. In some cases, a user may interact with the database controller 408. In other cases, the database controller 408 may operate automatically without user interaction. The database 414 may be an example of a single database, a distributed database, multiple distributed databases, a data store, a data lake, or an emergency backup database.

Memory 410 may include random-access memory (RAM) and read-only memory (ROM). The memory 410 may store computer-readable, computer-executable software including instructions that, when executed, cause the processor to perform various functions described herein. In some cases, the memory 410 may contain, among other things, a basic input/output system (BIOS) which may control basic hardware or software operation such as the interaction with peripheral components or devices.

The processor 412 may include an intelligent hardware device, (e.g., a general-purpose processor, a digital signal processor, a central processing unit (CPU), a microcontroller, an Application-Specific Integrated Circuit ASIC, a Field-Programmable Gate Array FPGA, a programmable logic device, a discrete gate or transistor logic component, a discrete hardware component, or any combination thereof). In some cases, the processor 412 may be configured to operate a memory array using a memory controller. In other cases, a memory controller may be integrated into the processor 412. The processor 412 may be configured to execute computer-readable instructions stored in a memory 410 to perform various functions (e.g., functions or tasks for personalized drug compounding based on AI-driven diagnostics and real-time data integration).

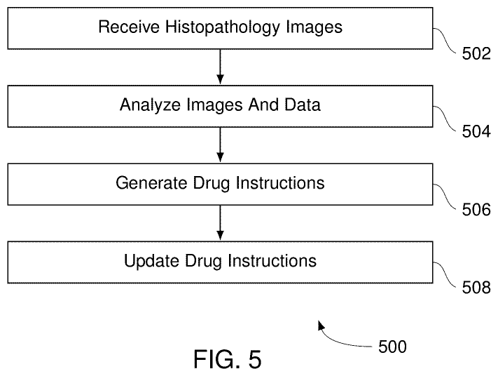

FIG. 5 shows a flowchart illustrating a method 500 for personalized drug compounding based on AI-driven diagnostics and real-time data integration in accordance with various aspects of the present disclosure. The operations of the method 500 may be implemented by one or more components of a networked computing system as described herein. For example, the operations of the method 500 may be performed by personalized drug compounding component 404 or through execution of machine-readable instructions 106 as described with reference to FIG. 4 and FIG. 1, respectively. In some examples, one or more components of a networked computing system may execute a set of instructions to control the functional elements of the component(s) to perform the described functions. Additionally or alternatively, the one or more components of a networked computing system may perform aspects of the described functions using special-purpose hardware.

At 502, the method 500 may include receiving, by a server, patient specimen and patient-specific clinical data including biomarker expressions, pharmacogenomic profiles, and organ function metrics. The operations of 502 may be performed in accordance with examples as disclosed herein. In some examples, aspects of the operations of 502 may be performed by a data receiving component 108 as described with reference to FIG. 1.

At 504, the method 500 may include analyzing, by the server, the patient specimen to extract tissue subtypes and biomarker features, and correlating the extracted features with the patient-specific clinical data to generate diagnostic outputs. The operations of 504 may be performed in accordance with examples as disclosed herein. In some examples, aspects of the operations of 504 may be performed by an image analysis component 110 and a diagnostic correlation component 112 as described with reference to FIG. 1.

At 506, the method 500 may include generating, by the server, personalized drug compounding instructions based on the diagnostic outputs and a clinical rule engine, the instructions specifying formulation types, dosages, and excipients tailored to the patient-specific clinical data. The operations of 506 may be performed in accordance with examples as disclosed herein. In some examples, aspects of the operations of 506 may be performed by a compounding instruction generation component 114 as described with reference to FIG. 1.

At 508, the method 500 may include updating, by the server, the personalized drug compounding instructions in response to changes in the patient-specific clinical data, the updates incorporating real-time adjustments to the formulation types, dosages, or excipients. The operations of 508 may be performed in accordance with examples as disclosed herein. In some examples, aspects of the operations of 508 may be performed by an instruction updating component 116 as described with reference to FIG. 1.

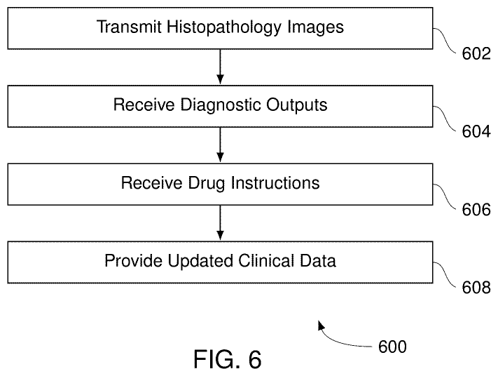

FIG. 6 shows a flowchart illustrating a method 600 for personalized drug compounding based on AI-driven diagnostics and real-time data integration in accordance with various aspects of the present disclosure. The operations of the method 600 may be implemented by one or more components of a networked computing system as described herein. For example, the operations of the method 600 may be performed by personalized drug compounding component 404 or through execution of machine-readable instructions 106 as described with reference to FIG. 4 and FIG. 1, respectively. In some examples, one or more components of a networked computing system may execute a set of instructions to control the functional elements of the component(s) to perform the described functions. Additionally or alternatively, the one or more components of a networked computing system may perform aspects of the described functions using special-purpose hardware.

At 602, the method 600 may include transmitting, by a user device, patient specimen and patient-specific clinical data including biomarker expressions, pharmacogenomic profiles, and organ function metrics to a server. The operations of 602 may be performed in accordance with examples as disclosed herein. In some examples, aspects of the operations of 602 may be performed by a data receiving component 108 as described with reference to FIG. 1.

At 604, the method 600 may include receiving, by the user device, diagnostic outputs generated by the server, the diagnostic outputs including tissue subtypes, biomarker features, and correlations with the patient-specific clinical data. The operations of 604 may be performed in accordance with examples as disclosed herein. In some examples, aspects of the operations of 604 may be performed by a diagnostic correlation component 112 as described with reference to FIG. 1.

At 606, the method 600 may include receiving, by the user device, personalized drug compounding instructions generated by the server based on the diagnostic outputs and a clinical rule engine, the instructions specifying formulation types, dosages, and excipients tailored to the patient-specific clinical data. The operations of 606 may be performed in accordance with examples as disclosed herein. In some examples, aspects of the operations of 606 may be performed by a compounding instruction generation component 114 as described with reference to FIG. 1.

At 608, the method 600 may include providing, by the user device, updated patient-specific clinical data to the server in response to changes in the patient's condition, the updated data enabling the server to generate real-time adjustments to the formulation types, dosages, or excipients. The operations of 608 may be performed in accordance with examples as disclosed herein. In some examples, aspects of the operations of 608 may be performed by an instruction updating component 116 as described with reference to FIG. 1.

In one aspect, the platform receives heterogeneous data inputs including histopathology images, clinical chemistry results, and patient electronic health record (EHR) data. These inputs are subjected to preprocessing and normalization modules that standardize and convert information into FHIR-compatible formats. The normalized data is supplied to an AI engine comprising convolutional neural networks and multimodal fusion models that extract patterns, detect biomarkers, and generate machine-learned features. A generative interface produces natural-language summaries of the AI outputs, while a rules engine applies compounding, dosing, and regulatory logic to ensure compliance.

The system generates personalized drug compounding instructions which are supported by a cloud deployment layer, a FHIR output formatter, and pharmacy integration services for automated batch record creation and audit logging. Outcome monitoring modules capture patient efficacy and adherence results which are routed into a model refinement engine for continuous learning and improvement, thereby closing the clinician-facing feedback loop.

In another aspect, the invention incorporates patient-facing features that extend the workflow to end users. A secure patient app or portal interfaces with an identity and consent service to enable authentication, authorization, and consent management. Patient-entered information and device data, obtained via a device and wearable gateway, are introduced into the preprocessing pipeline. Patients receive personalized instructions, adherence reminders, and educational materials through notification services and explainability modules that render plain-language summaries derived from the AI outputs. Patient outcomes and reported experiences are returned through the app into the outcome monitoring modules, ensuring that both human-reported and machine-detected signals are incorporated into the adaptive learning loop.

The disclosed system thereby establishes an end-to-end workflow from data ingestion, through AI analysis and regulatory validation, to automated compounding, pharmacy system integration, patient communication, and continuous refinement. The architecture provides a scalable, interoperable, and cloud-deployable framework suitable for centralized or field-based laboratory settings.

Hybrid and Multi-Model Approaches: To leverage the strengths of different methods, the predictive module can be implemented as a hybrid system that combines rule-based logic with learning-based models, or multiple model types together. In one embodiment, the module includes a rule-based expert system component that works in tandem with a machine learning (ML) model and a natural language processor NLP. The expert system may encode established medical protocols or safety rules. For example, “if the patient's kidney function is below a threshold, avoid Drug A” or “if diagnosis is Condition Q, first-line therapy is Drug X or Y.” These rules can serve as a first filter or a sanity check on the machine learning model's output. The hybrid workflow might operate as follows: the machine learning predictor (neural network or ensemble) generates a preliminary recommendation based purely on learning. Then the rule-based component evaluates that recommendation against its knowledge base.

If the ML recommendation violates a known medical rule or contradicts a well-established guideline, the system can override or adjust the suggestion. Alternatively, the rule-based system might narrow the field of options before the ML model runs. For example, feeding into the ML model only those treatment options that are considered permissible for the given context. This ensures that the final decision respects hard constraints and accumulated medical wisdom, while still benefiting from data-driven insights. Another hybrid approach is multi-phase inference: for example, first use a simpler interpretable model to get an initial guess or to compute intermediate features (like an estimated disease risk score), and then feed that result into a more complex model that finalizes the dosage or drug choice. Because the first model is simpler, it can provide an explanation or a level of confidence that can modulate the second. The predictive module in some embodiments might also run multiple models in parallel (each potentially using a different algorithm or trained on different data subsets) and then combine their outputs using a meta-decision logic. For instance, a rule-based model might output “Treatment A” while a neural network outputs “Treatment B;” the system could be designed to detect such conflicts and either defer to a human or use a tie-breaking logic (perhaps weighted by the confidence of each or by predefined priority of certain models). By blending approaches, the predictive module can achieve a balance of innovation and caution by using patterns from data to personalize care but always within safe bounds defined by medical science.

Model Training and Data Characteristics: The predictive module's efficacy heavily depends on how it is trained and the data it has seen. Training is generally performed on a large and diverse set of patient cases with known outcomes or known best treatments. These training cases might include historical electronic health records, clinical trial datasets, case reports, or any combination of diagnostic inputs paired with either the treatment that was administered or the treatment outcome (e.g., recovery, improvement metrics). For supervised learning models (neural networks, ensembles, etc.), the training data would have labels that could be the actual medication and dose a physician chose (particularly if over time those choices proved effective), or an outcome label that the model tries to predict (for instance, predict patient's blood pressure after 1 week if given Drug X vs. Drug Y, and the training process adjusts the model to better predict outcomes, indirectly teaching it which drug leads to better outcomes). The training data is ideally multimodal, for example, a given case might include numeric data (lab results), categorical data (diagnosis codes, demographics), free text (doctor's notes, which could be parsed by natural language processing to extract features), and even images or genomics. One challenge is ensuring the dataset is representative of the patient population and the decision space. If certain conditions or minorities are underrepresented, the model could initially be less accurate for them. Accordingly, the training process may include techniques for handling class imbalance and bias. For example, the training algorithm might upweight the importance of rare cases or incorporate synthetic data for underrepresented scenarios, ensuring the model learns to handle those adequately. Additionally, training the predictive module often involves cross-validation and testing on separate validation datasets to guard against overfitting (so that the model not only remembers training examples but generalizes correctly to new patients). In some embodiments, transfer learning can be applied. For example, if a neural network is first trained on a general medical decision task or a related problem (such as diagnosing diseases from symptoms), its internal layers might capture useful medical knowledge that can be repurposed and fine-tuned for the specific task of therapy recommendation in the DSP context. Reinforcement learning-based approaches may use simulated patient environments or retrospective data structured as state-action-reward trajectories. For instance, to train a model to recommend dosage adjustments over time, the historical progression of patients (with states being their health metrics at each visit and actions being dose changes) can serve as the experience from which the model learns a policy that maximizes cumulative health improvement. The model could also be initialized or bounded by pharmacological knowledge (for example, embedding known pharmacokinetic models into the training process, so that the AI learns representations consistent with how drugs behave in the body). Overall, the training regimen is designed to imbue the predictive module with a broad, nuanced understanding of how inputs relate to outputs, closely mirroring real-world clinical decision patterns.

Adapting to New Data (Continuous Learning): Medical knowledge and data are continuously evolving. As new studies emerge, new drugs become available, and the DSP platform itself will encounter novel patient scenarios once deployed. Therefore, the predictive module can be designed to adapt to new data over time. In one embodiment, the system periodically retrains or fine-tunes the model using newly collected case data from the platform's use (subject to appropriate data privacy protections and aggregation). For example, after the system has been in use for six months, it might have records of hundreds of patients, what the AI recommended, what was actually compounded and given (which could be the AI's suggestion or a human override), and how the patients fared. These can be added to the training set and the model retrained so that it learns from any mistakes and reinforces correct decisions. This could be done on a scheduled basis (e.g., monthly model updates) or triggered when sufficient new data is available or when performance monitoring indicates a drift (say the model's recommendations start differing from clinicians' choices in a systematic way, indicating some shift in practice or patient population).

In some advanced embodiments, online learning might be employed. The model updates incrementally case-by-case. For instance, after each patient encounter, the model slightly adjusts its parameters to better fit that data point. However, given the high stakes, online learning is typically used cautiously, updates might first occur in a shadow/clone model that is tested before replacing the main model, to ensure no destabilizing changes. Another mechanism for adaptation is active learning, where the model can identify cases where it's uncertain and explicitly request those be reviewed or added to its training (for example, if the model had low confidence on a particular recommendation and a clinician provided the solution, the system flags that scenario for the training pipeline with high importance).

If entirely new kinds of data become available (for instance, a new biomarker test is invented), the module can incorporate those by expanding its feature space and retraining the model to take advantage of them. Modular architectures facilitate this. For example, if the model has separate components for different data types, one can plug in a new sub-model for the new data source and combine it with the existing ones. The adaptability ensures that the predictive module remains up-to-date and continues to improve its accuracy as more cases are processed, effectively learning from the collective experience. To maintain safety, when the model is updated, it can be validated on a test set (including critical cases) and possibly compared against the prior model's decisions to verify that changes are beneficial (for example, confirming that no previously correct recommendation has been degraded).

Uncertainty Estimation and Handling: It is crucial in medical applications that the system not only makes a prediction but also provides insight into how much confidence to place in that prediction. The predictive module can thus be equipped with mechanisms to estimate and handle uncertainty in its inferences. In one embodiment, the model inherently produces a confidence score or probability distribution over possible outputs. For instance, if a neural network classifier is used, it might output a softmax probability for each treatment option (e.g., 90% confidence that Drug A is best, 10% for Drug B). This confidence information can be used by the system in several ways: the decision logic module could decide to only accept the AI's recommendation outright if the confidence is above a certain high threshold and otherwise flag the recommendation for human review or for additional data gathering. Alternately, the system might present the top N options to a clinician with their respective confidence levels, allowing the human to make the final call if it's a borderline case.

Beyond simple probability outputs, more sophisticated uncertainty quantification techniques may be utilized. In some embodiments, the predictive module is realized as a Bayesian neural network. Essentially a neural model that treats its weights as distributions rather than fixed values, yielding a distribution over outputs. When such a model evaluates an input, it can perform multiple forward passes sampling different weight configurations (often via Monte Carlo dropout or similar techniques) to see how much the outputs vary, a high variance indicates uncertainty. Another approach is to use an ensemble of models trained slightly differently (e.g., with different random initializations or subsets of training data) if all models agree on the recommendation, the system is likely certain, if they disagree, that's a sign of uncertainty. The predictive module can compute metrics like entropy of the output distribution or the gap between the top two recommendation scores as a measure of how decisive the prediction was.

Handling uncertainty also means gracefully degrading the system's behavior when confidence is low: instead of forcing a potentially incorrect precise recommendation, the module might output a broader suggestion or a request for help. For example, it could identify that the patient's data doesn't closely match anything in its training experience (perhaps an out-of-distribution case) and signal this condition. The system might then either default to a conservative treatment (like a broad-spectrum approach) and mark it for follow-up or directly ask for expert input. This way, the AI behaves safely by not overstepping when it's not sure. Incorporating uncertainty awareness tends to increase trust and safety and clinicians using the system can be alerted to pay extra attention when the AI itself is uncertain, analogous to how a human doctor might say “I'm not 100% sure, we might need more tests.” All such uncertainty-handling mechanisms are designed to ensure that the predictive module's output is not taken as gospel in every scenario, but rather weighted by its estimated reliability.

Explainability of Inferences: Given that medical decisions must often be audited and justified, the predictive module can support explainability features. This means that when it produces a recommendation, it can also provide information about the rationale behind that recommendation. If a rule-based or expert system component is involved, explanations can be relatively straightforward (e.g., “Rule 5 triggered because patient's liver enzyme was high, indicating dose reduction”). For learning-based models, which are more opaque, additional techniques are employed. In one embodiment, the system tracks feature importance for a given prediction. Essentially identifying which input factors (among the patient's data) most influenced the output. For example, a gradient-based attribution method might determine that the patient's extremely high cholesterol level was a major factor in the model choosing a certain drug-dosage combination aimed at reducing cardiovascular risk. The module can output a set of key factors like: “Recommended Drug A due to high cholesterol and presence of diabetes (these features strongly drove the decision).”

Another approach to explainability is using surrogate models or interpretable proxies. The predictive module might internally train an interpretable model (like a small decision tree or rule set) that approximates the behavior of the complex model around the region of the current input. This surrogate can then be presented as an explanation (for instance, a short rule: “IF biomarker X>1.2 and age >50 THEN use Drug A” could summarize why the complex model chose Drug A for this patient). In some cases, the module might also retrieve example-based explanations: it can find similar past cases in its training data to show as evidence (e.g., “Patient profile matches 85% with a previous patient who responded well to Drug A”). Yet another explainability feature is concept-based interpretation. For instance, the model might internally recognize a pattern corresponding to a known medical syndrome, and thus it can state the recommendation in those terms (“The combination of features suggests a high likelihood of Complication Z, hence the model recommends adding Drug B as precaution”). The patent disclosure earlier noted that trust in AI is bolstered if clinicians understand reasoning; hence, the predictive module can be configured to output metadata along with the recommendation, which the UI or decision logic layer can transform into a human-readable explanation. This fosters transparency, allowing medical professionals to validate and feel comfortable with the AI's role. Importantly, if an explanation reveals something questionable (say the model relied heavily on a feature that is a lab error or irrelevant), the clinician can catch that and adjust the course, providing an added layer of safety.

Safety Mechanisms and Fail-safes: In a high-stakes domain like personalized medicine, the predictive module must operate within strict safety and ethical boundaries. Several design features ensure that its suggestions do not compromise patient safety. First, constraint enforcement is often built into the module's logic or the surrounding system: certain outputs are disallowed or automatically adjusted. For example, if the model (left unchecked) were to suggest a morphine dose higher than a set safety limit, the system can automatically cap the dose to that limit or require human approval to exceed it. These constraints can be implemented as hard limits coded into the decision logic or as modifications to the model's loss function during training (so the model learns never to violate them). Second, the presence of the aforementioned rule-based overlay in hybrid systems acts as a safety net. Ensuring that well-known contraindications and precautions are heeded regardless of what pure data-driven inference might say. Third, the module's recommendations can be cross-checked against external databases in real time. In one embodiment, after the module outputs a recommendation, the system queries a drug-interaction database or clinical decision support database to verify there are no flags (e.g., if the patient is on medication M that wasn't part of the input feature set, and the AI recommends medication N that adversely interacts with M, this external check would catch it). If a conflict is found, the system can adjust the recommendation or at least alert a human. Another safety mechanism is stability and consistency checks: the system can monitor how much the recommendations change with minor fluctuations in input. If the predictive module's output is very sensitive (for example, a tiny change in one lab value drastically flips the recommendation), that might indicate an overfitting or unstable decision boundary. The module or a separate validation function can detect such conditions and either smooth the decision (choose a generally safe default) or ask for confirmation. Additionally, the development and validation process for the predictive module involves using hold-out test cases, simulation, and even clinical pilot studies to ensure its behavior aligns with standard of care. Any observed failure modes can be addressed by adjusting the model or adding new rules. Furthermore, the system can incorporate a “human-in-the-loop” safeguard: for particularly novel or critical decisions, the AI's output is not directly enacted without a sign-off. The predictive module might mark a recommendation as “standard” (safe to auto-compound) or “edge case” (needs review). This categorization itself can be learned or rule-based (for instance, if the confidence is low or if the recommended therapy is something rarely seen, default to requiring approval). Over time, as the model proves itself, these thresholds might be relaxed in certain domains, but the system always retains an override or stop mechanism. Lastly, accountability and traceability of the predictive module's decisions enhance safety. Each recommendation can be logged along with the input data and the model version. This means if any question arises later (say a patient had an adverse reaction), one can retrospectively analyze what the module recommended and why, facilitating audits and continuous improvement. In sum, the predictive modeling module is engineered not only for accuracy and efficiency but with robust safeguards so that its integration into medical decision-making upholds the paramount requirement: do no harm.

Interfaces and Integration with Other Components: The predictive module interfaces upstream with the data preprocessing/feature extraction module and downstream with the decision logic/output module, thus playing a pivotal middle role in the DSP pipeline. It expects as input a standardized patient data representation (which could be a vector, a structured object, or a set of values) produced by the preprocessing stage. This interface can be an API or function call within the software—for example, the feature extraction module may call a predict (patient_features) function of the inference module. In real-time operation, latency can be a consideration: if a deep neural network is large, the system might employ hardware acceleration (such as a GPU or an AI chip) to compute the inference within seconds. The module might also stream intermediate results. For instance, if the inference process is iterative (like an optimization algorithm running through multiple iterations to find the best compound proportions), it could update a state that the decision logic can monitor (though typically the decision logic waits for the final output). Once the predictive module determines a recommendation, it hands off that result to the decision logic/plan synthesis module. The interface here may include not just the raw recommendation (e.g., “Drug A, 100 mg”) but also ancillary info like the confidence level, or alternative options ranked, or explanatory factors, as described above. The decision logic module then uses this information to compose the detailed compounding instructions and check any final constraints. In some architectures, the predictive module and decision logic might be combined or overlap. For example, a single neural network could both decide the drug and format the instructions. However, separating them has advantages for clarity and maintainability (the predictive module focuses on what to give, and the decision logic focuses on how to prepare and deliver it).

The predictive module also can receive feedback inputs as part of its interface in a broader sense. For example, if the compounding execution or the clinician provides an override or adjustment, the decision logic or UI can feed that information back to the AI engine for learning purposes. During development or tuning phases, the module's interface may allow developers to input test cases or tweak parameters (like turning on/off certain rules or switching between model versions) to compare outputs. Additionally, the predictive module may interface with a monitoring system; for instance, a watchdog might query the module with known test inputs periodically to ensure its functioning (for reliability in a deployed setting).

In distributed deployments, the interface could even be a network call. For example, an edge device sends features to a cloud-hosted predictive service and receives back a prediction. All these interactions are designed to be seamless and fast enough to allow the DSP to function in near real-time, and they maintain data consistency (patient IDs, timestamps are passed along to ensure the recommendation corresponds to the correct individual and context). Thus, the predictive modeling/inference module, through its various internal mechanisms and careful integration, serves as the intelligent heart of the personalized compounding platform. Analyzing, learning, and proposing treatment decisions that the rest of the system can act on to deliver customized healthcare solutions.

It should be noted that the methods described herein describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and components described in connection with the disclosure herein may be implemented or performed with a general-purpose processor, a digital signal processor (DSP), an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A method for personalized drug compounding based on AI-driven diagnostics and real-time data integration, comprising:
receiving, by a server, patient specimen and patient-specific clinical data including biomarker expressions, pharmacogenomic profiles, and organ function metrics;
analyzing, by the server, the patient specimen to extract tissue subtypes and biomarker features, and correlating the extracted features with the patient-specific clinical data to generate diagnostic outputs;
generating, by the server, personalized drug compounding instructions based on the diagnostic outputs and a clinical rule engine, the instructions specifying formulation types, dosages, and excipients tailored to the patient-specific clinical data; and
updating, by the server, the personalized drug compounding instructions in response to changes in the patient-specific clinical data, the updates incorporating real-time adjustments to the formulation types, dosages, or excipients.

2. The method of claim 1, further comprising analyzing the patient-specific clinical data to identify contraindications or adverse interactions among potential excipients, active pharmaceutical ingredients, or delivery mechanisms, and incorporating the identified contraindications or interactions into the generation of the personalized drug compounding instructions.

3. The method of claim 1, further comprising applying an explainable AI framework to the diagnostic outputs to generate interpretability data that identifies key features influencing the personalized drug compounding instructions, the interpretability data being configured for review by clinicians or regulatory bodies.

4. The method of claim 1, further comprising integrating multi-modal data from histopathology images, structured clinical data, and unstructured electronic health record notes to enhance the diagnostic outputs and refine the personalized drug compounding instructions.

5. The method of claim 1, further comprising conducting a risk stratification analysis on the patient-specific clinical data to classify the patient into a treatment response group, the classification being used to adjust the formulation types, dosages, or excipients in the personalized drug compounding instructions.

6. The method of claim 1, further comprising incorporating reinforcement learning to evaluate longitudinal patient outcomes and dynamically update the clinical rule engine, the updates being used to refine the personalized drug compounding instructions based on observed therapeutic performance.

7. The method of claim 1, wherein the personalized drug compounding instructions specify excipient modifications based on the patient-specific clinical data, including allergy profiles, metabolic enzyme activity, or prior adverse reactions to excipients.

8. The method of claim 1, wherein the diagnostic outputs include a confidence score for each identified tissue subtype and biomarker feature, the confidence score being used to prioritize the generation of the personalized drug compounding instructions.

9. The method of claim 1, wherein the patient-specific clinical data includes therapeutic response history, and the personalized drug compounding instructions are adjusted to include alternative delivery mechanisms for patients with prior non-responsiveness to specific formulations.

10. The method of claim 1, wherein the real-time adjustments to the personalized drug compounding instructions include changes to the formulation types based on updated biomarker expression levels or newly identified tissue subtypes.

11. The method of claim 1, wherein the clinical rule engine incorporates evidence-based guidelines for rare diseases, and the personalized drug compounding instructions are tailored to include specialized formulations for patients with rare or atypical presentations.

12. A system configured for personalized drug compounding based on AI-driven diagnostics and real-time data integration, comprising:
a processor;
memory coupled with the processor; and
instructions stored in the memory and executable by the processor to cause the system to:
receive patient specimen and patient-specific clinical data including biomarker expressions, pharmacogenomic profiles, and organ function metrics;
analyze the patient specimen to extract tissue subtypes and biomarker features, and correlate the extracted features with the patient-specific clinical data to generate diagnostic outputs;
generate personalized drug compounding instructions based on the diagnostic outputs and a clinical rule engine, the instructions specifying formulation types, dosages, and excipients tailored to the patient-specific clinical data; and
update the personalized drug compounding instructions in response to changes in the patient-specific clinical data, the updates incorporating real-time adjustments to the formulation types, dosages, or excipients.

13. The system of claim 12, wherein the instructions are further executable by the processor to cause the system to analyze the patient-specific clinical data to identify contraindications or adverse interactions among potential excipients, active pharmaceutical ingredients, or delivery mechanisms, and incorporate the identified contraindications or interactions into the generation of the personalized drug compounding instructions.

14. The system of claim 12, wherein the instructions are further executable by the processor to cause the system to apply an explainable AI framework to the diagnostic outputs to generate interpretability data that identifies key features influencing the personalized drug compounding instructions, the interpretability data being configured for review by clinicians or regulatory bodies.

15. The system of claim 12, wherein the instructions are further executable by the processor to cause the system to integrate multi-modal data from histopathology images, structured clinical data, and unstructured electronic health record notes to enhance the diagnostic outputs and refine the personalized drug compounding instructions.

16. The system of claim 12, wherein the instructions are further executable by the processor to cause the system to conduct a risk stratification analysis on the patient-specific clinical data to classify the patient into a treatment response group, the classification being used to adjust the formulation types, dosages, or excipients in the personalized drug compounding instructions.

17. The system of claim 12, wherein the instructions are further executable by the processor to cause the system to incorporate reinforcement learning to evaluate longitudinal patient outcomes and dynamically update the clinical rule engine, the updates being used to refine the personalized drug compounding instructions based on observed therapeutic performance.

18. The system of claim 12, wherein the personalized drug compounding instructions specify excipient modifications based on the patient-specific clinical data, including allergy profiles, metabolic enzyme activity, or prior adverse reactions to excipients.

19. The system of claim 12, wherein the diagnostic outputs include a confidence score for each identified tissue subtype and biomarker feature, the confidence score being used to prioritize the generation of the personalized drug compounding instructions.

20. A non-transitory computer-readable medium storing code for personalized drug compounding based on AI-driven diagnostics and real-time data integration, the code comprising instructions executable by a processor to:
receive patient specimen and patient-specific clinical data including biomarker expressions, pharmacogenomic profiles, and organ function metrics;
analyze the patient specimen to extract tissue subtypes and biomarker features, and correlate the extracted features with the patient-specific clinical data to generate diagnostic outputs;
generate personalized drug compounding instructions based on the diagnostic outputs and a clinical rule engine, the instructions specifying formulation types, dosages, and excipients tailored to the patient-specific clinical data; and
update the personalized drug compounding instructions in response to changes in the patient-specific clinical data, the updates incorporating real-time adjustments to the formulation types, dosages, or excipients.
